(19) United States
(12) Patent Application Publication
(10) Pub. No.: US 2026/0248714 A1
(43) Pub. Date: Aug. 27, 2026

(54) METHOD OF COSMETIC TREATMENT FOR IMPROVING THE SKIN NIGHT-TIME RENEWAL PROCESS AND FOR PROTECTING THE SKIN FROM FREE RADICALS ASSOCIATED WITH BLUE LIGHT EXPOSURE

(71) Applicants: ISP INVESTMENTS LLC, Wilmington, DE (US); ELC MANAGEMENT LLC, MELVILLE, NY (US)

(72) Inventors: Florian LABARRADE, ANTIBES (FR); Audrey LE MESTR, ANTIBES (FR); Joël MANTELIN, CANNES (FR); Nadine PERNODET, Dix Hills, NY (US); Chia CHEN, Eastchester, NY (US); Jacqueline TRIVERO, Putnam Valley, NY (US)

(73) Assignees: ISP INVESTMENTS LLC, Wilmington, DE (US); ELC MANAGEMENT LLC, MELVILLE, NY (US)

(21) Appl. No.: 18/878,203

(22) PCT Filed: Jun. 27, 2023
(86) PCT No.: PCT/US2023/069159

§ 371 Date: Dec. 23, 2024

Related U.S. Application Data
(60) Provisional application No. 63/356,750, filed on Jun. 29, 2022.

(51) Int. Cl. A61K 8/9789; A61Q 17/04; A61Q 19/00; A61Q 19/02; A61Q 19/08

(52) U.S. Cl. CPC A61K 8/9789; A61Q 17/04; A61Q 19/00; A61Q 19/007; A61Q 19/02; A61Q 19/08; A61K 2800/522; A61K 2800/805

(57) ABSTRACT

Methods for improving the skin night-time renewal process, for protecting the skin from free radicals associated with blue light exposure, and for increasing the expression of the proteins Angel2 and Timeless include topically applying to the skin a cosmetic composition that has an active ingredient that include an aqueous extract of lavender aerial parts (Lavandula angustifolia).

### TECHNICAL FIELD

The invention belongs to the field of cosmetics and more particularly to a method of cosmetic treatment for improving the appearance of the skin.

The invention relates to a method of cosmetic treatment for improving the skin night-time renewal process, for ensuring correct DNA replication and DNA damage response and more particularly for increasing the expression of the proteins Angel2 and Timeless, and for protecting the skin from free radicals associated with blue light exposure.

### TECHNICAL BACKGROUND OF THE INVENTION

Many consumers are concerned with the appearance of their skin and the signs of skin aging, such as wrinkles and loss of firmness or freckles, age spots.

The skin is a vital organ composed of several layers (dermis, epidermal layers, and stratum corneum), which covers the entire surface of the body and provides protective, sensitive, immune, metabolic, or thermoregulatory functions. The skin, like the other organs, is subject to aging.

The skin is subjected to a complex physiological process of aging. Intrinsic or chronological aging is the consequence of a genetically programmed senescence and of biochemical alterations due to endogenous factors. In the skin, aging process is characterized by a slow-down in the regeneration of cells and extracellular matrices, a gradually loss of dermal collagen and elastic fibers resulting in dermal and epidermal atrophy, dryness, a reduction in elasticity and firmness of the skin, the appearance of fine lines and wrinkles hyperpigmentation or hypopigmentation blemishes, age spots, etc.

Extrinsic aging, on the other hand, is due to environmental attack such as pollution, sun light irradiation (including UV radiation), diseases, lifestyle, etc. This is superimposed on intrinsic aging at zones which are chronically exposed to these attacks; this is then known as photo-aging.

Angel2 is a member of the CCR4 family of deadenylases and catalyzes the removing of the poly(A) tail from an mRNA during RNA quality control (Godwin et al. Kiss your tail goodbye: the role of PARN, Nocturnin, and Angel deadenylases in mRNA biology. Biochim Biophys Acta. 2013; 1829(6-7):571-579). Angel2 not only prevents accumulation of nonfunctional RNAs, but also regulates normal mRNAs.

Angel2 is a rhythmic protein and shows a peak expression in the early night. Angel2, contributes to the regulation of diurnal and nocturnal gene expression pattern (Zhang, Ray, “Building and Mining an Atlas of the Mammalian Circadian Transcriptome” (2015). Publicly Accessible Penn Dissertations. 1171. http://repository.upenn.edu/edissertations/1171). Hundreds of genes were found to exhibit rhythmicity in poly(A) tail length, thus deadenylases play a critical role in regulating poly(A) tail rhythms and circadian gene expression (Yao et al. Critical role of deadenylation in regulating poly(A) rhythms and circadian gene expression. PLoS Comput Biol. 2020; 16(4): e1007842; Yao et al. Correction: Critical role of deadenylation in regulating poly(A) rhythms and circadian gene expression. PLoS Comput Biol. 2021 17(5): e1009065). The importance of endogenous rhythmicity transcription, and translation feedback loops of genes in skin physiology has been reviewed (Matsui et al. Biological Rhythms in the Skin. Int J Mol Sci. 2016; 17(6):801; Lyons et al. Circadian Rhythm and the Skin: A Review of the literature. J Clin Aesthet Dermatol. 2019; 12(9):42-45).

Angel2 possess a 2′,3′-cyclic phosphatase activity and may play a key role in regulating the response to cellular stress (Pinto et al. ANGEL2 is a member of the CCR4 family of deadenylases with 2′,3′-cyclic phosphatase activity. Science. 2020; 369(6503):524-530). RNA molecules are frequently modified with a terminal 2′,3′-cyclic phosphate group, Angel2 plays a key role during a surveillance mechanism known as ribosome-associated quality control (RQC) (Pinto et al. ANGEL2 is a member of the CCR4 family of deadenylases with 2′,3′-cyclic phosphatase activity. Science. 2020; 369(6503):524-530). Ribosomes that stall inappropriately during protein synthesis harbor truncated/misfolded polypeptides (Yip et al. Mechanism for recycling tRNAs on stalled ribosomes. Nat Struct Mol Biol. 2019; 26(5):343-349). Angel2 protects the cell from the production of proteotoxic polypeptides that can have deleterious effects (Pinto et al. ANGEL2 is a member of the CCR4 family of deadenylases with 2′,3′-cyclic phosphatase activity. Science. 2020; 369(6503):524-530).

Timeless is another essential protein for circadian rhythm, and it interacts with the circadian clock protein cryptochrome 2 (Unsal-Kacmaz et al., 2005. Coupling of Human Circadian and Cell Cycles by the Timeless Protein. Molecular and Cellular Biology. 3109-3116).

Timeless is a protein belonging to the “Replication Protection Complex” that ensures replication checkpoint and correct DNA replication.

DNA replication genes are upregulated in afternoon and evening (E Paatela, et al., Circadian Regulation in Tissue Regeneration. Int. J. Mol. Sci. 2019, 20, 2263).

It requires coordination between replication fork progression and deoxynucleotide triphosphate-generating metabolic pathways. Timeless and Tipin, another protein of the “Replication Protection Complex”, have roles in replication fork stabilization and sister chromatid cohesion. Depletion in Timeless has been shown to lead to a robust fork slowdown (Leman A R et al., 2009. Human Timeless and Tipin stabilize replication forks and facilitate sister-chromatid cohesion. Journal of Cell Science 123:660-670).

Timeless protein is also a core component of the cell cycle checkpoint system. It interacts with the cell cycle checkpoint proteins Chk1 and the ATR-ATRIP complex and plays an important role in the DNA damage checkpoint response. Down-regulation of Timeless in human cells seriously compromises replication and intra-S checkpoints, indicating an intimate connection between the circadian cycle and the DNA damage checkpoints that is in part mediated by the Timeless protein (Unsal-Kacmaz et al., 2005).

Blue light represents a portion of visible light between 400 and 495 nm. It is the most high-energy portion of the visible spectrum. Most of the blue light received originates from the sun. In our interior environment, we are increasingly exposed to artificial blue light from LEDs (Light-Emitting Diodes) present in lighting smartphone, tablet, computer and television screens.

Blue light has effects on the skin. It has been demonstrated on keratinocytes and endothelial cells of human skin that blue light exerts an anti-proliferative effect and promotes the differentiation of the keratinocytes.

Considering the harmful effects that blue light can produce on the skin, and more particularly DNA damages associated with free radical exposure, it seems important to be able to develop a cosmetic composition which can be used for protecting the human skin from blue light.

Peschel et al. (Current Biology 19, 241-247, Feb. 10, 2009) showed that in Drosophila, the clock factor Timeless is degraded after blue light, resulting in a daily reset and adaptation of the circadian clock to its environment.

Lavender, and in particular true lavender (Lavandula angustifolia), is a wild species native to Provence (south-eastern France) widely used to produce essential oils in Europe, North Africa, the Middle East, and Asia, particularly for the perfume industry.

The composition used in the present disclosure comprises as an active ingredient an aqueous extract of lavender aerial parts enriched with small RNAs with a length of at most 150 nucleotides, with sugars, with phenolic compounds and with organic acids, free of DNA, which was disclosed in the patent application WO2021156104 (also published as the U.S. patent application Ser. No. 17/795,389), incorporated herein by reference.

The above aqueous extract of lavender does not have the disadvantages of currently known extracts, namely a strong odour, an instability such as essential oil in formulas for topical application, an irritant or allergenic character due to the presence of terpene molecules such as linalool and does not require the use of detergents and solvents potentially toxic in cosmetics.

The above aqueous extract of lavender was designed for protecting the skin from external aggressions and oxidation, combating the signs of skin aging, increasing photoprotection, lightening the skin, improving skin hydration, reinforcing the barrier function, or soothing the skin and for improving biological mechanisms associated with skin repair during the night.

It was surprisingly found that a composition comprising the above aqueous extract of lavender has favorable effects with regard to its ability to ensure correct DNA replication and DNA damage checkpoint response, ensure RNA quality control check, rhythmic control of RNA poly(A) tail lengths and to participate in the ribosome-associated quality control pathway.

The present disclosure provides a method for improving the skin night-time renewal process, for protecting the skin from blue light exposure, and more particularly for increasing the expression of the proteins Angel2 and Timeless.

### SUMMARY OF THE INVENTION

The invention relates to a method for improving the skin night-time renewal process and for protecting the skin from free radicals associated with blue light exposure comprising topically applying to the skin a cosmetic composition comprising i) as an active ingredient, an aqueous extract of lavender aerial parts (Lavandula angustifolia) comprising, by weight of the total weight of the extract, 4 to 20 g/kg of dry weight, containing 0.5 to 7 g/kg of sugars, 200 to 1000 mg/kg of organic acids, 600 to 1200 mg/kg of phenolic compounds and 10 to 100 mg/kg of low molecular weight RNA with a length of at most 150 nucleotides, and ii) a physiologically acceptable medium.

The invention also relates to a method for improving the skin night-time renewal process comprising ensuring correct DNA replication and DNA damage response, ensuring RNA quality control check, rhythmic control of RNA poly(A) tail lengths, participating in the ribosome-associated quality control pathway and increasing the expression of the proteins Angel2 and Timeless.

The invention also relates to a method of protecting the skin from free radicals associated with blue light exposure comprising decreasing the level of free radicals and increasing the expression of the proteins Angel2 and Timeless in skin cells.

### BRIEF DESCRIPTION OF THE DRAWINGS

The invention and its advantages will be better understood from the following description and non-limiting embodiments, illustrated with reference to the attached figures, in which:

FIG. 1 shows microscopic observation of Angel2 staining on keratinocytes.

FIG. 2 shows microscopic observation of Timeless staining on keratinocytes.

FIG. 3 Normalized ROS Production in Keratinocytes after 30 minutes of Blue Light Irradiation.

FIG. 4 Normalized ROS Production in Keratinocytes after 1, 2, and 4 Hours of Blue Light Irradiation.

FIG. 5 Normalized ROS Production in Keratinocytes after 1, 2, or 4 Hours of Blue Light Irradiation with and without Lavender Extract at 1%.

FIG. 6 shows microscopic observation of Angel2 staining on keratinocytes after Blue Light Irradiation with and without Lavender Extract at 0.5%.

FIG. 7 shows microscopic observation of Timeless staining on keratinocytes after Blue Light Irradiation with and without Lavender Extract at 0.5%.

### DETAILED DESCRIPTION OF THE INVENTION

### Definitions

All terms used in this description have the most widely known meaning unless otherwise stated. For the purposes of the invention the following terms are defined as follows:

“Lavender” refers to the aerial parts of the species Lavandula angustifolia, i.e., lavender flowers and the small stems carrying the flowers.

The terms “small RNAs” or “low molecular weight RNAs”, or “small RNAs of a length of at most 150 nucleotides” means non-coding RNAs (ribonucleic acids) of small molecular weight, of a length of at most 150 nucleotides, such as all types of small non-messenger RNAs, single and/or double stranded, for example micro-RNAs, interfering RNAs, introns, small nuclear RNAs or any RNA fragment. Electrophoretic analysis shows that the small RNAs present in the lavender extract of the invention have various molecular weights of approximately 30 to 150 nucleotides.

“Organic acids” means α-hydroxy acids (or AHAs), i.e., carboxylic acids derived from fruit or plant sugars, such as glycolic, malic, citric, tartaric, succinic and uronic acids.

“Phenolic compounds” means molecules of plant origin that have an aromatic ring bearing one or more hydroxyl groups, such as phenolic acids, flavonoids or their derivatives. Phenolic compounds are known to be powerful antioxidant molecules.

“Sugars” mean monosaccharides, especially glucose and fructose, as well as oligo- and polysaccharides.

When a range of values is described, the limits of that range should be understood as explicitly including all intermediate values in the range. For example, a range of values between 1% and 10% should be understood to include 1%, 2%, 3%, 4%, 5%, 6%, 7%, 8%, 9%, and 10%, and also all decimal values between 1% and 10%.

Numerical percentage values are percentages by weight, i.e., the weight of a compound in relation to the total weight of the intended mixture, unless otherwise specified.

The compositions described in the present application may “comprise”, “consist of” or “consist substantially of” the essential compounds or optional ingredients.

“Consist substantially of” means that the composition or component may include additional ingredients, but only if the additional ingredients do not alter the basic or novel characteristics of the composition or use described in the present application.

A “physiologically acceptable medium” means a vehicle that is suitable for coming into contact with the outer layers of the skin or mucous membranes, without toxicity, irritation, undue allergic response and the like or intolerance reaction, and proportionate to a reasonable benefit/risk ratio.

“Topical application” means applying or spreading the aqueous extract enriched with small RNAs of a maximum length of 150 nucleotides, sugars, phenolic compounds and organic acids according to the invention, or a composition containing it, on the surface of the skin or a mucous membrane.

“Skin” refers to the skin including skin appendages (hair and body hair, eyebrow, eyelashes, nails).

“Skin night-time renewal process” has the meaning of the various biological mechanisms associated with the repair of the skin during the night, including at least: ensuring correct DNA replication and DNA damage response, ensuring RNA quality control check, rhythmic control of RNA poly(A) tail lengths, participating in the ribosome-associated quality control pathway and increasing the expression of the proteins Angel2 and Timeless.

It is understood that the invention relates to mammals and more particularly to human beings.

The present disclosure relates to a method for improving the skin night-time renewal process, for increasing the expression of the proteins Angel2 and Timeless, and protect the skin from free radicals associated with blue light exposure comprising topically applying to the skin a cosmetic composition comprising, i) as an active ingredient, an aqueous extract of lavender aerial parts (Lavandula angustifolia) comprising, by weight of the total weight of the extract, 4 to 20 g/kg of dry weight, containing 0.5 to 7 g/kg of sugars, 200 to 1000 mg/kg of organic acids, 600 to 1200 mg/kg of phenolic compounds and 10 to 100 mg/kg of low molecular weight RNA with a length of at most 150 nucleotides and free of DNA, and ii) a physiologically acceptable medium.

### Extract of Lavender

The extraction process of the aerial parts of lavender (Lavandula angustifolia) was disclosed in the patent application U.S. Ser. No. 17/795,389, herein incorporated by reference.

Briefly, the process comprises the following steps:
a) bringing the lavender aerial parts into contact with water;
b) adding phytic acid at a concentration of between 1 and 10 mM and preferably at a concentration of 3 mM, to the mixture obtained in a) at a pH of between 10 and 11 and keeping under agitation at least 1 hour, at a temperature between 2° and 80° C.;
c) purifying the mixture obtained in b) by eliminating the aerials parts of the residual solid lavender, so as to recover soluble part which constitutes the crude aqueous extract;
d) filtering the aqueous extract by successive sequential filtrations on filters with decreasing porosity in order to clarify the plant extract until a filtration at 1 μm, to remove the residual solid part and collect the filtrate
e) adjusting the pH of the filtrate obtained in d) to a value between 6 and 8;
f) diluting the obtained extract in e) with a physiologically acceptable solvent, in order to increase the stability and the conservation of the extract over time.
g) checking the pH and readjusting it if necessary to a value between 6 and 7;
h) submitting the extract to low temperature sterilization to avoid any contaminations.

The diluted extract may be further preserved using any physiologically acceptable preservative.

This process yields an extract comprising, by weight of the total weight of the extract, 4 to 20 g/kg of dry weight, containing 0.5 to 7 g/kg of sugars, 200 to 1000 mg/kg of organic acids, 600 to 1200 mg/kg of phenolic compounds and 10 to 100 mg/kg of low molecular weight RNA with a length of at most 150 nucleotides, and ii) a physiologically acceptable medium.

The extract obtained by this process is free of DNA.

An example of the above disclosed process comprises:

In the step a), 35 g of dried lavender, ground into powder having a particle size ranging from 500 μm to 1 mm, are mixed in 963 g of distilled water;
in step b) 2 g/L or 3 mM of phytic acid are added. The pH is adjusted to 10.8 for optimal enrichment of the extract with low molecular weight RNAs and the mixture is then heated for 1 hour at 80° C. with stirring;

In a step c), after this hour of extraction, the mixture obtained in b) is filtered through filters with a pore size of 30 μm to remove the solid matter;
in step d) filtrations of the extract are carried out by lowering the filtration threshold from 50 to 20 μm then from 1 to 0.1 μm;
in step e), the pH is checked and then adjusted with an HCl solution, to pH values between 6 and 6.5 to preserve the small RNAs in the extract, and preferably the pH is adjusted at 6.3;
in step f) the dilution is carried out with plant-derived glycerine to obtain a final concentration of 30% glycerine and 70% lavender extract and preserved with the addition of 1.5% of phenoxyethanol;
in step g), the pH is controlled and adjusted using hydrochloric acid (HCl) to values comprised between 8 and 6.3;
in a step h) the diluted extract is submitted to low temperature sterilization for 8 hours at 72° C.

The obtained diluted extract has a dry weight of 9.5 g/kg and has a concentration of 3.7 g/kg total sugars, 698 mg/kg total organic acids, 1040 mg/kg total phenolics and 64 mg/kg low molecular weight RNAs with a length of at most 150 nucleotides.

The lavender extract disclosed herein thus comprises a wide range of phytomolecules that may have beneficial effects on the skin, without presenting a risk of skin irritation or other health damage.

### Composition

The cosmetic composition used in the present disclosure comprises i) as an active ingredient, an aqueous extract of lavender aerial parts (Lavandula angustifolia) comprising, by weight of the total weight of the extract, 4 to 20 g/kg of dry weight, containing 0.5 to 7 g/kg of sugars, 200 to 1000 mg/kg of organic acids, 600 to 1200 mg/kg of phenolic compounds and 10 to 100 mg/kg of low molecular weight RNA with a length of at most 150 nucleotides, and ii) a physiologically acceptable medium.

Advantageously, the extract of lavender aerial parts obtained by the process described above is added to the composition at a concentration of 0.5 to 2% by weight in relation to the total weight of the composition.

The composition used in the present disclosure may be applied by any suitable route and the formulation of the compositions will be adapted by the person skilled in the art.

Preferably, the compositions used in the present disclosure are in a form suitable for topical application. These compositions must therefore contain a physiologically acceptable medium, i.e., compatible with the skin and the skin appendages, without any risk of discomfort during their application and must cover all suitable cosmetic forms.

The compositions for implementing the invention may in particular be in the form of an aqueous, hydroalcoholic or oily solution, an oil-in-water emulsion, water-in-oil emulsion, or multiple emulsions; they may also be in the form of suspensions, or powders.

The compositions for implementing the invention may in particular be suitable for application to the skin, mucous membranes, lips and/or hair.

These compositions may be more or less fluid and may also have the appearance of a cream, lotion, milk, serum, ointment, gel, paste or foam. They may also be in solid form, such as a stick, or may be applied to the skin in the form of an aerosol.

Examples of physiologically acceptable media commonly used in the envisaged field of application are adjuvants necessary for the formulation, such as solvents, thickeners, diluents, antioxidants, colouring agents, sun filters, self-tanning agents, pigments, fillers, preservatives, perfumes, odour absorbers, essential oils, vitamins, essential fatty acids, surfactants, film-forming polymers, etc.

Advantageously, the composition according to the invention may comprise, in addition to the active ingredient according to the invention, at least one other active agent having cosmetic effects similar and/or complementary to those of the invention.

For example, the additional active agent(s) may be selected from: anti-aging, firming, lightening, moisturising, draining, microcirculation promoting, exfoliating, desquamating, extracellular matrix stimulating, energy metabolism activating, antibacterial, antifungal, soothing, anti-free radical, anti-UV, anti-acne, anti-inflammatory, anaesthetic, warm feeling inducing, cool feeling inducing, and slimming.

### Method of Cosmetic Treatment

The present disclosure relates to a method of cosmetic treatment for improving the skin night-time renewal process and for protecting the skin from free radicals associated with blue light exposure comprising topically applying to the skin a cosmetic composition comprising i) as an active ingredient, an aqueous extract of lavender aerial parts (Lavandula angustifolia) comprising, by weight of the total weight of the extract, 4 to 20 g/kg of dry weight, containing 0.5 to 7 g/kg of sugars, 200 to 1000 mg/kg of organic acids, 600 to 1200 mg/kg of phenolic compounds and 10 to 100 mg/kg of low molecular weight RNA with a length of at most 150 nucleotides, and ii) a physiologically acceptable medium.

In a particular embodiment the above method comprises applying a cosmetic composition comprising the aqueous extract of lavender aerial parts (Lavandula angustifolia) obtained by the process described above in the present disclosure.

In a particular embodiment the above method comprises applying a cosmetic composition comprising from 0.5 to 2% by weight in relation to the total weight of the composition of an aqueous extract of lavender aerial parts (Lavandula angustifolia).

In a particular embodiment, the present disclosure also relates to a method for improving the skin night-time renewal process wherein the skin night-time renewal process comprises ensuring correct DNA replication and DNA damage response, ensuring RNA quality control check, rhythmic control of RNA poly(A) tail lengths, participating in the ribosome-associated quality control pathway, and increasing the expression of the proteins Angel2 and Timeless, comprising topically applying to the skin a cosmetic composition comprising the aqueous extract of lavender of the present disclosure.

In another particular embodiment, the present disclosure also relates to a method for protecting the skin from free radicals associated with blue light exposure wherein the level of free radicals is decreased and the level of proteins Angel2 and Timeless is increased in skin cells, comprising topically applying to the skin a cosmetic composition comprising the aqueous extract of lavender of the present disclosure.

The extract of lavender aerial parts of the invention has been tested on key biological markers associated with night-time skin repair mechanisms, with correct DNA replication and DNA damage response.

More particularly, the extract of lavender aerial parts of the invention has been tested on the expression of the proteins Angel2 and Timeless, and for the protection against free radicals associated with blue light exposure.

The extract of lavender aerial parts of the invention demonstrated favorable effects with regard to its ability to ensure correct DNA replication and DNA damage checkpoint response, to ensure RNA quality control check, rhythmic control of RNA poly(A) tail lengths and to participate in the ribosome-associated quality control pathway.

### EXAMPLES

By way of illustration, exemplary embodiments of the process according to the invention are described below.

### Example 1: Evaluation of the Aqueous Lavender Extract on Angel2 Expression in Human Keratinocyte

### Principle:

The aim of this study is to show the effect of the application of the aqueous lavender extract versus a conventional extract of lavender on Angel2 expression in epidermal keratinocytes.

### Protocol:

Normal human keratinocytes were treated twice a day with a solution of 0.5% vol/vol of an aqueous lavender extract prepared according to the process described in the present disclosure or with a solution of 0.5% vol/vol of a conventional lavender extract for 48 hours. For immunolabelling by anti-Angel2 antibody, the cells were washed and fixed with 3.7% paraformaldehyde for 15 minutes at room temperature. Cellular membranes were permeabilized with a 0.2% Triton X-100 (Sigma) solution, for 10 minutes. Unspecific binding sites were blocked with 1% BSA (Sigma) solution for 30 minutes. The cells were then incubated in the presence of a specific anti-Angel2 antibody (Sigma, ref. HPA030796, rabbit polyclonal), and then a secondary suitable antibody, coupled with a fluorescent dye. After mounting in a particular medium, the slides were observed by epifluorescence microscope (Nikon Eclipse 80i microscope). Fluorescence intensity was quantified by analyzing the image using Volocity 6.3. software.

The conventional extract was prepared for comparative purpose from lavender aerial parts put in water in the same ratio than the extract of the present disclosure for one hour at room temperature under gentle agitation. The obtained mixture was submitted to sequential filtration to clarify the extract from 30 μm until sterilizing filtration of 0.2 μm, the pH was adjusted between 6.0 and 6.5.

### Results:

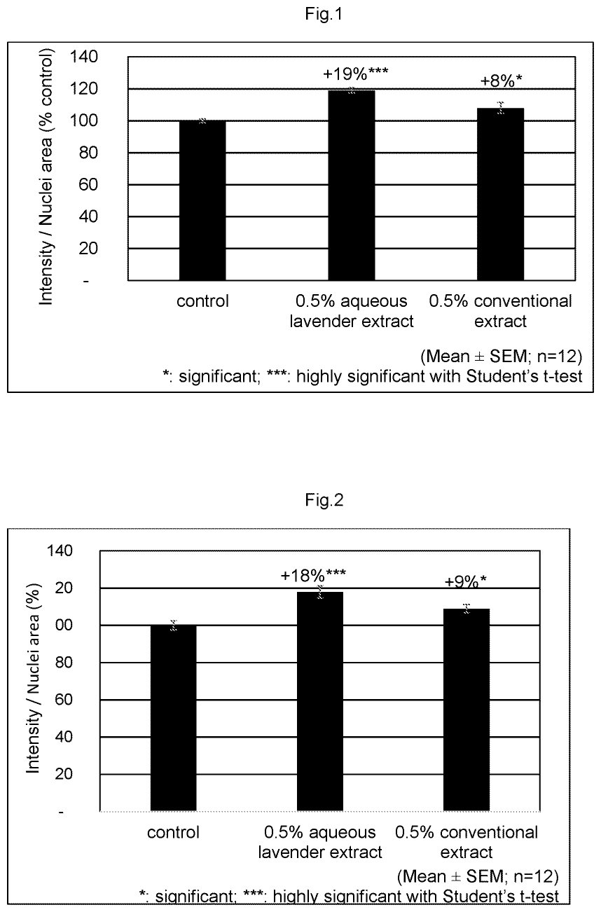

Microscopic observation showed a significantly highly (Student's t test) increase in Angel2 staining on keratinocytes, after the treatment with the aqueous lavender extract. The conventional extract showed a significant lower activity than the aqueous lavender extract. The results are illustrated in FIG. 1

TABLE 1
Quantification of the microscopic observations
of Angel2 Expression on Keratinocytes

| | Green intensity in nuclei/ |
|---|---|
| Conditions | nuclei area (%) |
| Untreated | 100 |
| 0.5% aqueous lavender extract | 119*** |
| 0.5% conventional extract | 108* |

*significant;
***highly significant with Student's t-test

### Conclusion

In vitro studies showed that a 48-hour application of 0.5% aqueous lavender extract of the invention was associated with an increase in Angel2 protein expression in cell nuclei, with a better efficacy than with a conventional lavender extract.

### Example 2: Evaluation of the Aqueous Lavender Extract on Timeless Expression in Human Keratinocyte

### Principle:

The aim of this study is to show the effect of the application of the aqueous lavender extract versus a conventional extract of lavender on Timeless expression in epidermal keratinocytes.

### Protocol:

Normal human keratinocytes were treated twice a day with a solution of 0.5% vol/vol of the aqueous lavender extract prepared according to the process described in present disclosure or the with a solution of 0.5% vol/vol of a conventional lavender extract for 48 hours. The preparation of the conventional lavender extract is described in example 1. For immunolabelling by anti-timeless antibody, the cells were washed and fixed with cold methanol for 4 minutes at 4° C. Unspecific binding sites were blocked with 1% BSA (Sigma) solution for 30 minutes. The cells were then incubated in the presence of a specific anti-Timeless antibody (Abcam, ref. ab72458, rabbit polyclonal), and then a secondary suitable antibody, coupled with a fluorescent dye. After mounting in a particular medium, the slides were observed by epifluorescence microscope (Nikon Eclipse 80i microscope). Fluorescence intensity was quantified by analyzing the image using Volocity 6.3. software.

### Results:

Microscopic observation showed a significantly highly (Student's t test) increase in Timeless staining on keratinocytes, after the treatment with the aqueous lavender extract. The conventional extract showed a significant lower activity than the aqueous lavender extract. The results are illustrated in FIG. 2.

TABLE 2
Quantification of the microscopic observations
of Timeless Expression on Keratinocytes

| | Green intensity in nuclei/ |
|---|---|
| Conditions | nuclei area (%) |
| Untreated | 100 |
| 0.5% aqueous lavender extract | 118*** |
| 0.5% conventional extract | 109* |

*significant;
***highly significant with Student's t-test

### Conclusion

In vitro studies showed that a 48-hour application of 0.5% the aqueous lavender extract was associated with an increase in Timeless protein expression in cell nuclei, with a better efficacy than a conventional extract.

### Example 3: Normalized ROS Production in Keratinocytes after 30 Minutes of Blue Light Irradiation

Protocol—30 minutes Experiment

Normal Human Epidermal Keratinocytes (NHEK, 45-year-old, Life Technologies; Catalog #A13401; Lot #2068397) were seeded at 40,000 cells per well into 2-4 well chamber slides with fully supplemented media (Epilife; Life Technologies; Catalog #MEPI500CA and HKGS; Life Technologies; Catalog #S0015). Plated cells were left to incubate overnight at standard conditions (37° C. and 5% CO2).

Following overnight incubation, cells were treated with a solution of 1% (vol/vol) of Lavender (aqueous lavender extract prepared according to the process described in the present disclosure and similar to the lavender extract used in Examples 1 and 2) in fully supplemented Media.

Post 24 hours, cells were washed twice with Dulbecco's Phosphate Buffer Solution (DPBS; Corning; Catalog #21-031-CV), and then replenished with 250 μl of DPBS with Glucose Supplement (Life Technologies; Catalog #14287-080).

Afterwards, one chamber slide was kept in complete darkness, while the other chamber slide went into the Blue Light chamber, where it was irradiated with 100 J/cm² of Blue Light (efflux 410 nm light source) (410 nm) (a duration of 30 minutes).

Post 30 minutes irradiation, cells were immediately stained for ROS and nuclei using CellROX Oxidative Stress Green Reagent (488) and HCS NuclearMask Blue Stain (Life Technologies; Catalog #H10325). Add 250 μl of CellROX and 250 μl of HCS NuclearMask to each well and let sit in incubator for 30 minutes at 37° C.

For CellROX a final concentration of 5 μM was given to the cells

For HCS Nuclear Mask, a final dilution of 5 μl of stain to 10 ml of complete media was given to the cells.

Once time has elapsed, remove media, wash once with DPBS and proceed to imaging and analysis on the Keyence BX-7. (ROS was taken in the 488 nm filter, and DAPI was taken in the 405 nm filter).

After all images were taken, they were analyzed on Nikon Elements for ROS production and cell counts.

Cell counts values were taken through object count function in Nikon Elements

ROS Production values were found by setting a binary threshold based on the intensity of the signal, and then taking the Sum Intensity of the binary threshold, which is automatically given through Nikon Elements.

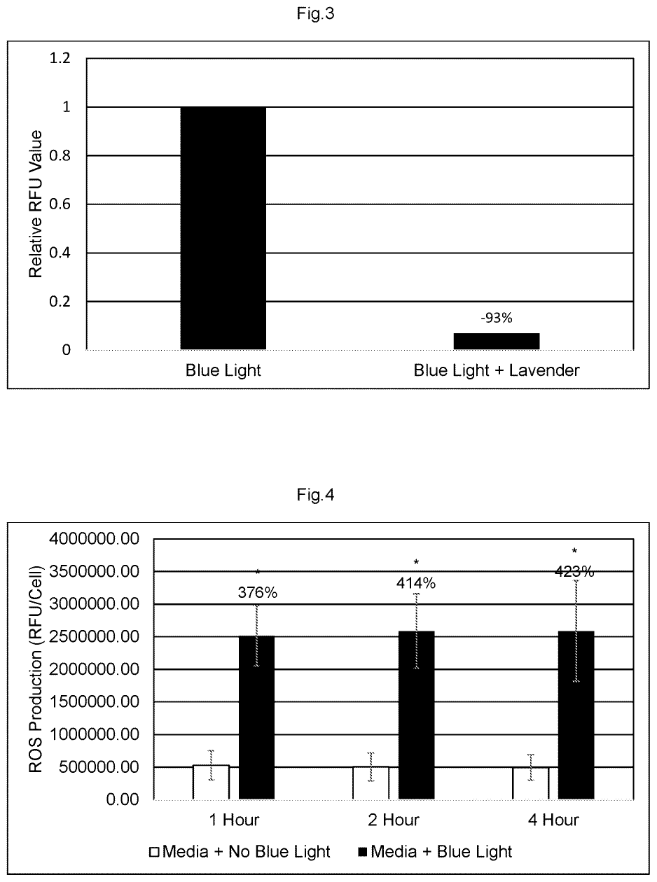

Results are illustrated at FIG. 3 and show 93% fewer free radicals with treatment of the aqueous Lavender extract at 1%.

### Example 4: Normalized ROS Production in Keratinocytes after 1, 2, and 4 Hours of Blue Light Irradiation

### Protocol: Multiple Blue Light Time Exposures

Normal Human Epidermal Keratinocytes (NHEK, 45-year-old, Life Technologies; Catalog #A13401; Lot #2068397) were seeded at 40,000 cells per well into 6-4 well chamber slides with fully supplemented media (Epilife; Life Technologies; Catalog #MEPI500CA and HKGS; Life Technologies; Catalog #S0015). Plated cells were left to incubate overnight at standard conditions (37° C. and 5% CO2).

Following overnight incubation, cells were washed twice with Dulbecco's Phosphate Buffer Solution (DPBS; Corning; Catalog #21-031-CV), and then replenished with 250 μl of DPBS with Glucose Supplement (Life Technologies; Catalog #14287-080).

Afterwards, three chamber slides were kept in complete darkness, while the other chamber slides went into the Blue Light chamber, where it was irradiated with Blue Light (efflux 410 nm light source) (410 nm) for a duration of 1, 2, and 4 hours.

Post each blue light irradiation time point, cells were immediately stained for ROS and nuclei using CellROX Oxidative Stress Green Reagent (488 nm) and HCS NuclearMask Blue Stain (Life Technologies; Catalog #H10325). Add 250 μl of CellROX and 250 μl of HCS NuclearMask to each well and let sit in incubator for 30 minutes at 37° C.
a. For CellROX a final concentration of 5 μM was given to the cells.
b. For HCS Nuclear Mask, a final dilution of 5 μl of stain to 10 ml of complete media was given to the cells.

Once time has elapsed, media was removed, cells were washed once with DPBS and imaging and analysis were processed on the Keyence BX-7. (ROS was taken in the 488 nm filter, and DAPI was taken in the 405 nm filter).

After all images were taken, they were analyzed on Nikon Elements for ROS production and cell counts.
c. Cell counts values were taken through object count function in Nikon Elements.
d. ROS Production values were found by setting a binary threshold based on the intensity of the signal, and then taking the Sum Intensity of the binary threshold, which is automatically given through Nikon Elements.

Results are illustrated at FIG. 4 and show blue light exposure creates very high level of free radical production.

### Example 5: Normalized ROS Production in Keratinocytes after 1, 2, or 4 Hours of Blue Light Irradiation with and without Lavender Extract

### Protocol: Multiple Blue Light Time Exposures and Treatment

Normal Human Epidermal Keratinocytes (NHEK, 45-year-old, Life Technologies; Catalog #A13401; Lot #2068397) were seeded at 40,000 cells per well into 9-4 well chamber slides with fully supplemented media (Epilife; Life Technologies; Catalog #MEPI500CA and HKGS; Life Technologies; Catalog #S0015). Plated cells were left to incubate overnight at standard conditions (37° C. and 5% CO2).

Following overnight incubation, 3 slides were treated with a solution of 1% (vol/vol) of Lavender (aqueous lavender extract prepared according to the process described in the present disclosure and similar to the lavender extract used in Examples 1 and 2) in fully supplemented Media.

Following overnight incubation, cells were washed twice with Dulbecco's Phosphate Buffer Solution (DPBS; Corning; Catalog #21-031-CV), and then replenished with 250 μl of DPBS with Glucose Supplement (Life Technologies; Catalog #14287-080)

Afterwards, three chamber slides were kept in complete darkness, while the other chamber slides went into the Blue Light chamber, where it was irradiated with Blue Light (efflux 410 nm light source) (410 nm) for a duration of 1, 2, and 4 hours. Post each blue light irradiation time point, cells were immediately stained for ROS and nuclei using CellROX Oxidative Stress Green Reagent (488 nm) and HCS NuclearMask Blue Stain (Life Technologies; Catalog #H10325). Add 250 μl of CellROX and 250 μl of HCS NuclearMask to each well and let sit in incubator for 30 minutes at 37° C.
a. For CellROX a final concentration of 5 μM was given to the cells.
b. For HCS Nuclear Mask, a final dilution of 5 μl of stain to 10 ml of complete media was given to the cells.

Once time has elapsed, media was removed, cells were washed once with DPBS and imaging and analysis were processed on the Keyence BX-7. (ROS was taken in the 488 nm filter, and DAPI was taken in the 405 nm filter).

After all images were taken, they were analyzed on Nikon Elements for ROS production and cell counts.
c. Cell counts values were taken through object count function in Nikon Elements.
d. ROS Production values were found by setting a binary threshold based on the intensity of the signal, and then taking the Sum Intensity of the binary threshold, which is automatically given through Nikon Elements.
e. Statistics were run using One-Way ANOVA with a Dunnett's Post Test. * if P<0.05.

Results are illustrated at FIG. 5 and show pre-treatment with 1% Lavender extract yields a free radical fighting action.

### Example 6: Evaluation of the Aqueous Lavender Extract on Angel2 Expression in Human Keratinocyte after Blue Light Irradiation

### Principle:

The aim of this study is to show the effect of the application of the aqueous lavender extract on Angel2 expression in epidermal keratinocytes after Blue Light Irradiation.

### Protocol:

Normal human keratinocytes were treated twice for 24 hours with a solution of 0.5% vol/vol of an aqueous lavender extract prepared according to the according to the process described in the present disclosure for 24 hours. Cells were then irradiated with blue light (415 nm) for a duration of 30 min and treated again twice for 24 hours. For immunolabelling by anti-Angel2 antibody, the cells were washed and fixed with 3.7% paraformaldehyde for 15 minutes at room temperature. Cellular membranes were permeabilized with a 0.2% Triton X-100 (Sigma) solution, for 10 minutes. Unspecific binding sites were blocked with 1% BSA (Sigma) solution for 30 minutes. The cells were then incubated in the presence of a specific anti-Angel2 antibody (Sigma, ref. HPA030796, rabbit polyclonal), and then a secondary suitable antibody, coupled with a fluorescent dye. After mounting in a particular medium, the slides were observed by epifluorescence microscope (Nikon Eclipse 80i microscope). Fluorescence intensity was quantified by analyzing the image using Volocity 6.3. software.

### Results:

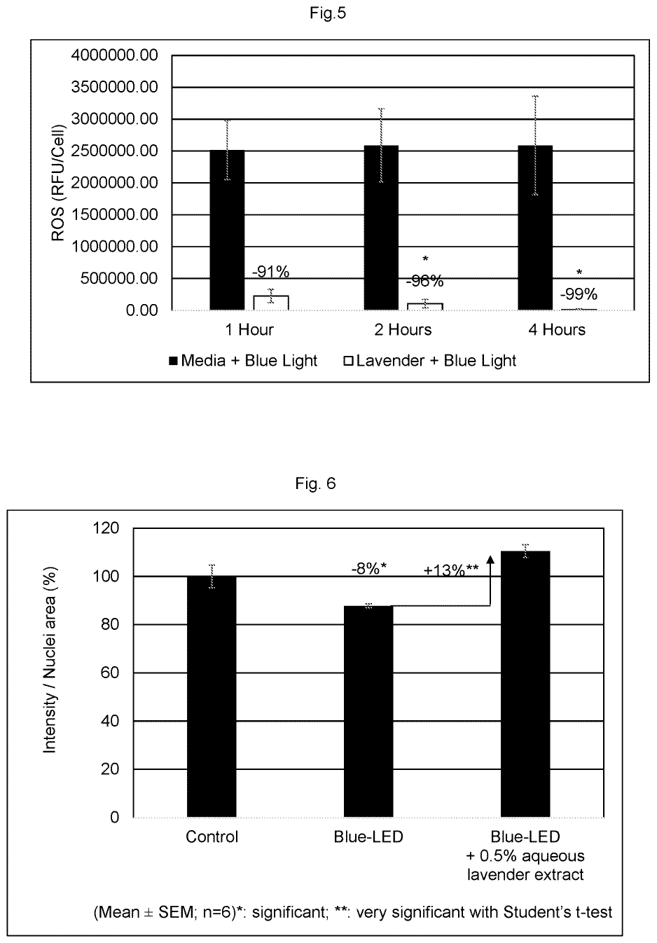

Microscopic observation showed a significant (Student's t test) decreased in Angel2 staining on keratinocytes following blue light irradiation. The treatment with the aqueous lavender extract allowed to increase Angel2 in stressed cells. The results are illustrated in FIG. 6.

TABLE 3
Quantification of the microscopic observations of Angel2
Expression on Keratinocytes after Blue Light Irradiation

| | Green intensity in nuclei/ |
|---|---|
| Conditions | nuclei area (%) |
| Control | 100 |
| Blue light | 92* |
| 0.5% aqueous lavender extract + blue light | 104** |

*significant;
**very significant with Student's t-test

### Example 7: Evaluation of the Aqueous Lavender Extract on Timeless Expression in Human Keratinocyte after Blue Light Irradiation

### Principle:

The aim of this study is to show the effect of the application of the aqueous lavender extract on Timeless expression in epidermal keratinocytes. After Blue Light Irradiation.

### Protocol:

Normal human keratinocytes were treated twice for 24 hours with a solution of 0.5% vol/vol of an aqueous lavender extract prepared according to the process described in the present disclosure for 24 hours. Cells were then irradiated with blue light (415 nm) for a duration of 30 min and treated again twice for 24 hours. For immunolabelling by anti-timeless antibody, the cells were washed and fixed with cold methanol for 4 minutes at 4° C. Unspecific binding sites were blocked with 1% BSA (Sigma) solution for 30 minutes. The cells were then incubated in the presence of a specific anti-Timeless antibody (Abcam, ref. ab72458, rabbit polyclonal), and then a secondary suitable antibody, coupled with a fluorescent dye. After mounting in a particular medium, the slides were observed by epifluorescence microscope (Nikon Eclipse 80i microscope). Fluorescence intensity was quantified by analyzing the image using Volocity 6.3. software.

### Results:

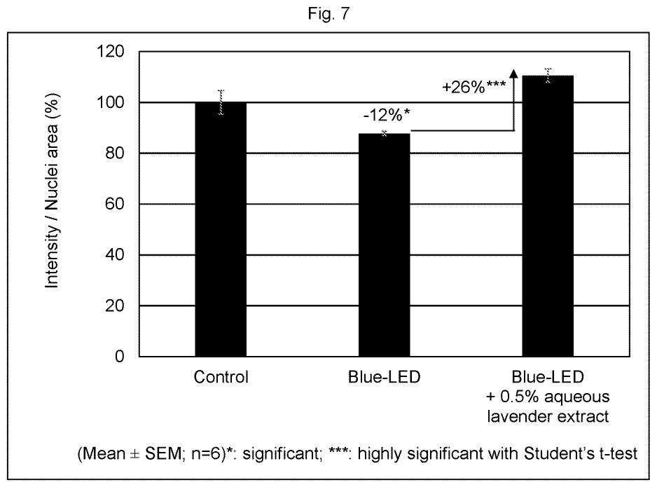

Microscopic observation showed a significant (Student's t test) decreased in Timeless staining on keratinocytes following blue light irradiation. The treatment with the aqueous lavender extract allowed to increase Timeless in stressed cells. The results are illustrated in FIG. 7.

TABLE 4
Quantification of the microscopic observations of Timeless
Expression on Keratinocytes after Blue Light Irradiation.

| | Green intensity in nuclei/ |
|---|---|
| Conditions | nuclei area (%) |
| Control | 100 |
| Blue light | 88* |
| 0.5% aqueous lavender extract + blue light | 111*** |

*significant;
***highly significant with Student's t-test

1. A method for improving the skin night-time renewal process and for protecting the skin from free radicals associated with blue light exposure, comprising topically applying to the skin a cosmetic composition comprising i) as an active ingredient, an aqueous extract of lavender aerial parts (Lavandula angustifolia) comprising, by weight of the total weight of the extract, 4 to 20 g/kg of dry weight, containing 0.5 to 7 g/kg of sugars, 200 to 1000 mg/kg of organic acids, 600 to 1200 mg/kg of phenolic compounds and 10 to 100 mg/kg of low molecular weight RNA with a length of at most 150 nucleotides, and ii) a physiologically acceptable medium.

2. The method of claim 1, wherein the aqueous extract of lavender (Lavandula angustifolia) is obtained by a process comprising the following steps
a) bringing the lavender aerial parts into contact with water;
b) adding phytic acid at a concentration of between 1 and 10 mM to the mixture obtained in a) at a pH of between 10 and 11 and keeping under agitation at least 1 hour, at a temperature between 2° and 80° C.;
c) purifying the mixture obtained in b) by eliminating the residual solid lavender, so as to recover soluble part which constitutes the crude aqueous extract;
d) filtering the aqueous extract obtained in step d) by successive sequential filtrations on filters of decreasing porosity until a filtration at 1 μm;
e) adjusting the pH of the filtrate obtained in d) to a value between 6 and 8;
f) diluting the extract obtained in e) with a physiologically acceptable solvent;
g) checking the pH and readjusting it if necessary to a value between 6 and 7;
h) submitting the extract to low temperature sterilization.

3. The method of claim 2, wherein in step b) the phytic acid is at a concentration of 3 mM.

4. The method of claim 1, wherein the skin night-time renewal process comprises ensuring correct DNA replication and DNA damage response, ensuring RNA quality control check, ensuring rhythmic control of RNA poly(A) tail lengths, participating in the ribosome-associated quality control pathway, and increasing the expression of the proteins Angel2 and Timeless.

5. The method of claim 1, wherein protecting the skin from free radicals associated with blue light exposure comprises the level of free radicals associated with blue light exposure is decreased and the level of proteins Angel2 and Timeless is increased in skin cells.

6. The method of claim 1, wherein the cosmetic composition comprises from 0.5 to 2% of the aqueous extract of lavender aerial parts (Lavandula angustifolia), by weight of the total weight of the composition.

7. The method of claim 1, wherein the cosmetic composition is in the form chosen among an aqueous solution, a hydroalcoholic solution, an oily solution, an oil-in-water emulsion, a water-in-oil emulsion, a multiple emulsion, a suspension or a powder.

8. The method of claim 1, wherein the cosmetic composition is suitable for application to the skin, mucous membranes, and lips.
